(19) United States
(12) Patent Application Publication
(10) Pub. No.: US 2010/0233269 A1
(43) Pub. Date: Sep. 16, 2010

(54) MINERALIZED POLYMER PARTICLES AND THE METHOD FOR THEIR PRODUCTION

(75) Inventors: Katarzyna Gorna, Vienna (AT); Andreas Goessl, Vienna (AT)

Correspondence Address: Townsend and Townsend and Crew LLP, Two Embarcadero Center, Eighth Floor, San Francisco, CA, 94111-3834, US

(73) Assignees: Baxter International, Inc., Deerfield, IL (US); Baxter Healthcare S.A., Glattpark (Opfikon) (CH)

(21) Appl. No.: 12/722,639

(22) Filed: Mar. 12, 2010

Related U.S. Application Data
(60) Provisional application No. 61/159,922, filed on Mar. 13, 2009.

(51) Int. Cl. A61K 9/14; A61K 33/24; A61K 33/32; A61K 33/26; A61K 33/04; A61K 33/00; A61K 33/38; A61P 19/00

(52) U.S. Cl. 424/489; 424/617; 424/641; 424/646; 424/630; 424/702; 424/722; 424/618; 424/653

(57) ABSTRACT

A process for mineralizing a particulate organic material includes providing particles of a non-porous, swellable organic material, and contacting the particles with a solution containing at least one cation and a solution containing at least one anion, thereby obtaining the mineralized particulate organic material.

### FIELD OF THE INVENTION

The present invention relates to the field of preparing materials, suitable for use as bone filling material. The materials comprise an organic polymer and a mineral phase.

### BACKGROUND OF THE INVENTION

Critical size bone defects resulting, for example, from injury or tumour resection, do not heal spontaneously. The use of autologous bone (autograft) from the patient has obvious disadvantages. Only limited amount of bone graft can be harvested and this procedure results in high morbidity of the donor site. For this reason there is a need for an efficient bone graft substitute material that can overcome the limitations of autografting. Typically, bone graft materials are produced from inorganic material such as hydroxyapatite or tri-calcium phosphate, or mixtures thereof. Calcium phosphate (CaP) materials possess excellent biocompatibility and good osteoconductive properties. They are produced as powders, granules or porous blocks. CaP powders is not the best material to be used in bone regeneration indications. Optimal bone substitute scaffold should have a specific porosity to allow vascularization and new bone in-growth. On the other hand, there is a problem with shaping CaP granules or blocks while filling bone defects. Pure CaP materials are brittle and typically have a low tensile and compressive strength. Furthermore, upon implantation small particles are produced that are known to be detrimental for bone regeneration and can potentially migrate through the vascular system in uncontrolled manner. Also degradation time of porous granules or blocks of CaP sometimes is not optimal to match the regeneration and remodelling process of new bone.

Bone typically consists of 22 wt.-% of collagen fibers with 69 wt.-% of homogenously distributed hydroxyapatite crystals with specific chemistry, morphology and size. From a material science point of view, bone is an example of a natural composite material with excellent integrity between organic and inorganic phases.

Therefore, various attempts have been made to artificially mimic the properties of natural bone in bone substitute materials. The U.S. Pat. No. 5,455,231, for example, discloses mineralized collagen obtainable from solubilised collagen contacted with calcium phosphate solution under basic conditions. In this approach the whole mineralization process is done in one step by progressive addition of calcium and phosphate solution to the dispersion/solution of collagen. The CaP salt slowly precipitates on the collagen fibers as the concentration of calcium and phosphate ions increases with addition of salt solution. The main drawback of this method is the very high pH of the process that is required when higher content of mineral phase needs to be obtained. Inorganic materials synthesized at high pH have obvious limitation as internal implants. Simple washing in order to reduce the material pH is not effective and in consequence there is a risk of surrounding tissue damage by local increase of pH after implantation.

One of the advantages of using composite materials in the bone regeneration process is their superior mechanical properties over purely ceramic material. Furthermore, the presence of inorganic particles in the organic matrix usually accelerates degradation process of all implants.

It is a challenge to create materials that can mimic the natural composition of bone matrix with homogeneously located inorganic crystals. A lot of effort has gone into integrating the organic matrix with inorganic material in order to achieve implants with properties like those of native bone.

Bone substitute materials that are usually produced by simply mixing of organic matrix with CaP powder suffer from very weak integration between organic and inorganic phases. After implantation, such materials are mechanically weak, and undergo. quick and uncontrolled resorption. Another limitation of the traditional composites for bone regeneration is the use of CaP crystals with coarse particles having sharp edges that are potentially harmful for surrounding tissue, in addition to a high propensity to create fine particles that are known to be detrimental for bone formation.

Other methods on composite materials containing natural polymers and various phases of CaP employ a biomimetic approach in their formation. For this method of pre-mineralization using simulated body fluid (SBF) to deposit salts onto carrier substrate materials. This approach is time consuming, as a concentration of calcium and phosphate ions in SBF is very low. Also, it requires material with specific surface composition to induce spontaneous nucleation of the mineral phase.

A very complicated process for the production of mineralized gelatin as a bone graft material has been reported in patent WO 2007/101171. This method involves isolation of gelatin from the human cortical or cancellous bone in such a way that the mineral component is still presented in the gelatin. At the same time the DBM (de-mineralized bone matrix) is isolated from the bone and both components, DBM and mineralized gelatin, are combined into the bone graft substitute material. The mineralization process described in this patent is complicated; another negative aspect of this method is that there is a potential risk of virus transmission related to the internal implants produced from animal or human driving components.

The WO 2003/089022 discloses a collagen composite with an amorphous liquid phase as mineral phase as mineralizing solution. This mineralizing solution is constituted by an acidic polymer such as poly-L-aspartic acid or polyacrylic acid and calcium containing material. The polymer mediates coating of minerals onto the organic collagen substrate. It is one goal of the present invention to prevent the inclusion of such acidic polymers into bone substitute material and maintain a high chemical resemblance to natural bone.

Yaylaoglu et al. (Biomaterials, 1999, 20, 1513-1520) describe the preparation of a mineralized lyophilized collagen sponge, which can be used as an osteochondral implant. The process includes a stepwise formation of calcium phosphate crystals over a period of time of 12 to 24 hours on this porous material.

### SUMMARY OF THE INVENTION

The subject of the invention are particles with a mineral phase distributed on their surfaces and inside the particles, as well as a method for making such particles comprising an intimate composite of the organic-inorganic phases.

In one aspect the present invention provides a process for mineralizing a particulate organic material comprising
a) providing particles of a non-porous, swellable organic material,
b) providing a solution of containing at least one cation,
c) providing a solution of containing at least one anion,
d) in any subsequent order:
   contacting said particles with said solution of the cation for a time period allowing adsorption of said cation on said organic material,
   contacting said particles with said solution of the anion for a time period allowing adsorption of said anion on said organic material,
      thereby obtaining said mineralized particulate organic material.

In this invention the mineral deposit is made on the surface and within the bulk of the organic particles by alternating treatment of particles in solutions of cations and anions leading to a situation where cations and anions are present within the material at concentrations above the solubility limit, leading to precipitation on the surface and within the organic particles. In contrast to the U.S. Pat. No. 5,455,231 mentioned above, it is possible (but not necessary) to use both solutions at physiological pH.

In another aspect the invention provides particles of mineralized organic material obtainable by said method.

The invention further provides particles of mineralized organic material comprising crystals of an anion and a cation, which crystals cover the organic material.

The main application for such pre-mineralized particles can be in bone regeneration. Therefore, the present invention provides in another aspect a method of treating a bone deficiency such as loss of bone matter, including surgical or natural loss, comprising applying to site of deficiency a fluid comprising particles of mineralized organic material, comprising crystals of an anion and a cation, which crystals cover the organic material.

### DETAILED DESCRIPTION OF THE INVENTION

The object of the invention are pre-mineralized particles of a non-porous, swellable organic material as a new material for tissue regeneration and methods for their preparation and application. The particles of the organic phase material can, e.g., be collagen, gelatin, fibrin, dextrane, agarose, hyaluronic acid, cellulose, fibronectin, a synthetic polymer, or a derivative thereof, but other kinds of organic material can be used as well.

A “derivative thereof” may be the substance in a crosslinked form or it may be a derivatized material obtained (intentionally or unintentionally) by any chemical or physical means. This shall also include truncations or cleavage of the molecules as well as the addition or removal of atoms, side chains, charges, etc.
Preferably the organic material is gelatin, collagen or fibrin. The particulate organic material is a non-porous and swellable material.
Non-porous means that the material is essentially free of pores or holes in the bulk and on the surface of the material, with the exception of unintentionally included small pores from e.g. residual air bubbles or for other reasons, filling only a marginal portion of the total volume.
Swellable means that the organic material has the capability to take up fluid material between 150 to 1000% and more of its own original weight.
The organic material used according to the present invention is hydrophilic and preferably crosslinked, e.g. crosslinked gelatin in particulate form.
The organic material can be biodegradable, being suitable for biological decomposition in vivo, or bioresorbable, i.e. a material able to be resorbed in vivo. Full resorption means that no significant extracellular fragments remain. A biodegradable material differs from a non-biodegradable material in that a biodegradable material can be biologically decomposed into units which may either be removed from the biological system and/or chemically incorporated into the biological system.

In this invention the mineral deposit can be made on the surface and within the bulk of particles of an organic material by alternating treatment in solutions of a cation and an anion. Both solutions can be prepared at physiological pH corresponding to natural bone conditions. It is also possible to perform the process at more basic conditions. Higher pH of solutions will accelerate the mineralization process. The pH of either or both of the cation or anion solution may be at least 3, at least 4, at least 5, at least 6, at least 6.5, at least 7 and/or up to 13, up to 12, up to 11, up to 10, up to 9, up to 8.5, or up to 8. Preferred pH ranges of either or both of the cation or anion solution are neutral, between 5 and 10, preferably between 6 and 9 or between 7 and 8. Likewise, it is preferred if the particulate organic material reacts pH neutral in contact with an aqueous solvent.

The solvent of either or both of the cation and anion solutions may be any solvent able to contact the organic material so that the cation and/or anions are able to crystallize on the organic material. In preferred embodiments the solvent is aqueous, i.e. containing water, or pure water, i.e. no other liquid solvent is present. Solvents may comprise water, alcohol, including methanol, ethanol, propanol, butanol, isopropanol, ketones, such as acetone, DMSO, etc. The solvent may or may not further comprise further compounds such as additives, emulsifiers, detergents and wetting agents. If such compounds are used, it is preferred that they are water removable and do not form stable and remaining complexes with the cation and anion crystals or the organic material. The additional compounds may be acidic, preferably neutral or basic.

Organic particles tend to swell in aqueous solutions, and small cations and anions can easily penetrate into the bulk of the material, where at the optimal concentrations crystals nucleate and progressively grow with time during the mineralization process.

The cation/anion solutions can be of any salts. These ions are preferably small molecules with low valence. Preferably the cation is at least divalent. It may be selected from inorganic ions like calcium, magnesium, barium, strontium, zinc, nickel, manganese, copper, selenium, iron, silicon, vanadium, silver, gadolinium, bismuth, or combinations thereof. One example of a solution of a cation may comprise calcium acetate, calcium chloride, calcium nitrate, calcium sulfate or other calcium salts. Calcium, as the main bone forming mineral is preferred for the inventive uses. For more specialized purposes other cations may be selected, e.g. strontium for increased radio-opacity and bioactivity. Strontium is known to be active in bone metabolism. Preferably the cation is selected from the group consisting of calcium, magnesium, barium and strontium.

Preferably the cation is selected from the group consisting of calcium, magnesium, barium and strontium and at least one further cation selected from the group consisting of zinc, nickel, manganese, copper, selenium, iron, silicon, vanadium, silver, gadolinium, bismuth is present.

Likewise, the anion may have a low valence, e.g. 1 to 6, preferably the anion is mono-, di-, tri-, tetra-, penta- or hexa-valent. The anion may be an inorganic or a small C₁—C₁₀ organic compound and may be selected, e.g., from phosphate, carbonate, sulfate, oxalate, silicate, fluoride, citrate, or combinations thereof. Example solutions of the anion may comprise sodium phosphate, potassium phosphate or other phosphate salts. For bone grafting phosphate is a preferred anion. For more specialized uses different anions may be selected such as carbonate increasing the solubility of the inorganic phase. Carbonate, for example, would likely lead to a more soluble mineral phase and faster degradation of the composite material.

The cations and anions to form crystals on the organic material are usually present in the solutions in concentrations so that the resulting salts are insoluble enough to precipitate on and inside the particles. In order to prevent premature precipitation the cation solution is preferably of a different salt than the solution of the anion. This salt may have a higher solubility than a salt formed by the cation and the anion. Likewise, the anion solution is preferably of a different salt than the solution of the cation. This salt may have a higher solubility than a salt formed by the cation and the anion.

To ensure constant growth of the mineral phase (cation/anion crystals) in a relatively short time it is important to keep the organic particles in solutions with concentrations of cation and anion ions above the saturation level at which there is spontaneous nucleation and subsequent cation-anion crystals growth. Otherwise, the mineral phase will not nucleate on a surface with low affinity to undergo spontaneous mineralization, or the initially deposited inorganic phase that is not fully transformed into crystalline material may re-dissolve.

On the other hand, to avoid uncontrolled precipitation of cation-anion crystals in the mineralized medium instead of on/in the organic substrate, the concentration of the cation and the anion ions should not be excessively high. For this reason the proposed process is used, characterized by subsequent treatment of the organic material particles first in a cation containing solution and then (optionally after washing with water) in an anion solution. Therefore, in preferred embodiments of the invention the particles are contacted with the solution of the cation before being contacted with the solution of the anion. In some embodiments the cations and/or anions, in their respective solutions, can be in concentrations of e.g. at least 95%, at least 98%, at least 100%, at least 105%, or at least 110%, and/or up to 180%, up to 160%, up to 140%, or up to 120%, of the quantitative solubility of a cation-anion solution (even though the solutions according to the invention are not used as such a mixed solution of the ions to be deposited).

To increase the inorganic phase of the mineralized product the contacting steps of the organic particles with the cation and/or anion solution(s) may be repeated, optionally multiple times. E.g. the subsequent contacting steps (in alternating order) may be repeated at least 2 times, 3, 4, 5, 6, 7, 8, 9, 10, 11, 12, 13, 14, 15, 16, 17, 18, 19, 20, 21, or at least 22 times the contacting steps might even be repeated up to 100 times or more, preferably up to 80 times.

Between or after each contacting step it is also possible to remove remaining and unbound cations, or anions, respectively. Preferably the inventive method comprises a washing step, wherein unbound anion is removed from the particles after contacting with said solution of the anion or wherein unbound cation is removed from the particles after contacting with said solution of the cation, respectively.

The rate at which the mineral phase will precipitate and grow can be affected by the volume ratio between mineralization medium and the organic matrix which is the subject of this process. It is advantageous that the volume of the mineralization medium is higher than the volume of organic material. For example, two parts of cation/anion solution can be used to mineralize one part of (swollen) solid organic material.

The cation or anion solutions are contacted with the organic material particles for a time sufficient to deposit said cations, or anions, respectively, on the organic material. Preferably the cation or anion solutions are contacted with the organic material particles for a time between 10 min and 120 min, preferably between 10 min and 60 min, more preferred about 30 min.

As starting reaction it is preferred to deposit the cation from its solution. Any subsequent deposition steps will result of crystal formation on any previously deposited cations or anions leading to mineral growth. As an example, in one mineralization cycle organic particles are suspended in the cation containing solution, and they are kept in this solution under gentle agitation for about 30 min in order to achieve equilibrium between cation concentration in the medium and in the swollen particles. Next, the cation solution is removed by careful centrifugation, and the organic particles are washed once with distilled water. The water is again removed by centrifugation and the organic particles are then suspended in the anion containing solution for about 30 min. During this time the anions migrate into the swollen structure of the particles and react with the cations that are still present inside the matrix to give cation-anion crystals. After this time the anion containing solution is removed by centrifugation and particles are washed at least once with distilled water that is subsequently removed by centrifugation. The procedure described above refers to one cycle of mineralization. The content of mineral phase increases with numbers of mineralization cycles.

This mineralization process can be carried out at room temperature but also at lower (close to 0° C.) or higher temperature (close to 50° C.) and any temperature range in between as well as even lower or higher temperatures. Thus, in preferred embodiments any one of the contacting steps is performed above −4° C., above −2° C., above 0° C., above 10° C., above 15° C. and/or below 75° C., below 70° C., below 50° C., or below 40° C. A preferred temperature range is between 0° C. and 50° C. or between 4° C. and 60° C.

The temperature may affect the type of mineral phase that precipitates, and also can have effect on the stability of organic material (matrix). Generally, the temperature should not accelerate organic material degradation as long as this degradation process is not intended.

Using this method, it was possible to deposit the mineral phase in amounts ranging from a few, up to 90 wt %, or more of the final total mass. The content of mineral phase increases with time of mineralization process, specifically, with numbers of cycles. The mineral (crystallized cation/anion) content may be up to 30%, up to 40%, or up to 50%, up to 60%, up to 70%, up to 80%, up to 90%, up to 100%, up to 110%, up to 120%, up to 130%, up to 140%, up to 150%, up to 175% or up to 200% of the mass of the organic material. The mineral content may be at least 1%, at least 5%, at least 10%, at least 20%, at least 30%, at least 40% or at least 50% of the mass of the organic material.

The type of the mineral phase can be modulated depending on the type of salt used in the process as well as by the ratio between cation to anion. Additionally, crystal morphology from needle-like to more spherical can be controlled by the pH and temperature of the process. Those skilled in the field can select appropriate parameters for any given cation/anion combination or deduce appropriate levels from known solubility parameters.

By choosing appropriate process conditions (e.g. temperature), it is possible to create particles that are highly porous throughout the bulk of the particle. This likely occurs when a solid inorganic layer on the outside of the particles holds the swollen volume of the particles, and the organic material is partially degraded during the process time. Such porous particles can be beneficial in applications where a high inner surface area is required, such as the seeding of cells on these particles.

The process conditions can also be tailored such that no degradation of organic material occurs. This yields particles with a higher organic content that retain more of the original properties of the organic matrix (e.g. swelling, rheology).

The particles can also be used to incorporate or bind drugs, bioactive substances selected from a group of substances consisting of a coagulation factor (e.g. thrombin, snake venoms, e.g. Ecarin, factor VII, factor VIII), a growth factor, a bone morphogenic protein, a hormone, a cytokine and a chemokine, or a gene or a gene construct encoding a bioactive molecule into the material by simply swelling the particles in a solution of the substances to be loaded, or by any other loading method. These drugs and substances will then be included in the inorganic (crystal) phase of the particles. Subsequently, the inventive particles may be used for the delivery of said drug or substance.

The mineralized particles can be used in dry or hydrated, but preferably in hydrated form or fluidized form to generate materials for tissue regeneration. The (mineralized or non-mineralized precursor) particles may be swollen or not. Preferably, the organic material is swellable from 150% up to 1000%, e.g. 200% to 500%, of its original weight in dry state. The particles may be provided in any fluid in which the particles are dispersed but water is preferred.

The particles of the organic phase material can, e.g., be collagen, gelatin, fibrin, dextrane, agarose, hyaluronic acid, cellulose, fibronectin, a synthetic polymer, or a derivative thereof, but other kinds of organic material can be used as well. Preferably the material is gelatin, collagen or fibrin. Preferably, it is water-insoluble, biodegradeable and bioresorbable. Natural gelatin is cheap and broadly available and can be obtained from many sources. Gelatin is a hydrolysate of collagen. Convenient animal sources of gelatin and collagen include chicken, turkey, bovine, porcine, or equine sources. The collagen can also be artificial or recombinant collagen. Preferably, the gelatin is crosslinked to prevent complete solubility. Crosslinking may be achieved by incomplete hydrolysation of collagen or chemical crosslinking using crosslinking reagents such as formaldehyde or divalent aldehydes.

In another aspect the present invention provides particles of mineralized organic material obtainable by the inventive method of subsequently using cation and anion solutions to deposit the crystal phase. Such particles are preferably microparticles being in the size range of micrometers. In preferred embodiments the particle size is 2000 μm or below. Even smaller particles may be used, e.g. of an average size below 1000 μm, below 500 μm, below 100 μm, below 50 μm, below 25 μm, below 10 μm, below 5 μm, below 1 μm, below 900 nm, below 500 nm, below 100 nm. The particles size may be at least 1 μm, at least 900 nm, at least 500 nm, at least 100 nm, or at least 10 nm. Preferably the particles have an average diameter of between 100 nm and 2000 μm.

One advantage of the inventive particles is that, after being formed from pH neutral solutions, the mineralized organic material will also react pH neutral in contact with an aqueous solvent. “pH neutral” is considered to increase, or alternatively also decrease, the pH of not more than 1, preferably not more than 0.5 For such a test e.g. equal amounts (mass) of particles and water, preferably without buffer substances, can be used.

In general, the invention provides particles of mineralized organic material comprising crystals of an anion and a cation, which crystals cover the organic material. Before or after the crystallization process the particles may have the above mentioned sizes. All embodiments as described above in relation to the inventive method, such as selection of specific cations or anions, of course also apply to the inventive particles.

In further embodiments the final particles, together with the inorganic crystalline phase, may be provided in fluidized form. A fluid, such as a suspension, dispersion, gel or paste of the particles allows easy application of the flexible product onto any bone graft site to be treated. The fluid may also be used as a glue in order to engulf larger scaffold-like bone grafts which are usually used in larger bone depleted regions. In order to form said fluid from other gels, or gel-forming compounds or (viscous) liquids can be used.

The inventive particles or the fluids comprising the same can be provided as a pharmaceutical composition for therapeutic application, in particular to treat bone defects. The compositions can take the form of a putty that can be molded and shaped to fit the exact form of the defect. Such a pharmaceutical composition has good injectability and moldability and hence can be used e.g. as a bone void filler.

Preferably the pharmaceutical composition comprises the particles according to the present invention and at least one additive selected from the group consisting of collagen, gelatin, fibrin, dextrane, agarose, hyaluronic acid, cellulose, fibronectin, a synthetic polymer or a derivative thereof.
If a more cohesive material is desired, the particles can be combined with other materials to achieve this objective. Examples of such additives can be selected from a group of materials consisting of modified cellulose, in particular methoxy cellulose, hydroxyethyl cellulose, hydroxypropyl cellulose, carboxymethyl cellulose, carboxymethyl cellulose sodium salt, carboxypropyl cellulose, alginic acid, alginic acid sodium salt, carrageenan, polyethylene glycol, carbomer, starch and its derivatives, hyaluronic acid and its derivatives, agar and its derivatives, guar gum, locust bean gum, xanthan gum, soluble gelatin, collagen, fibrin, furcellaran, etc. This combination can be achieved by simple mixing, or by coating the mineralized particles with the additive, e.g. in a fluid bed coating process. Also, depending on the formulation such composite materials can be made as an injectable material that fills a defect and subsequently sets in situ to the solid composite. The additive may be in any suitable concentration for an application of choice. Usually the additive is about 1 to 100%-wt. of the mineralized organic material. For other applications concentrations of about 2 to 50%-wt or 2 to 25%-wt might be preferred. The composition or particles may be used as implant material. The fluid or the composition may have a pH of at least 3, at least 4, at least 5, at least 6, at least 6.5, at least 7 and/or up to 13, up to 12, up to 11, up to 10, up to 9, up to 8.5, or up to 8.

In another utilization of these mineralized particles, the organic matrix can be removed by a suitable process (e.g. enzymatic or hydrolytic degradation or heat treatment to achieve thermal decomposition) leaving behind an inorganic structure with potentially unique structure and properties. Also, before this decomposition of the organic matrix the particles can be pressed into suitable 3D objects (e.g. disks) under high pressure, yielding three-dimensional inorganic materials with potentially unique structures and properties.

In another aspect the present invention relates to a method of treating a bone deficiency, a method for bone regeneration, tissue regeneration, comprising applying to site of deficiency a fluid comprising particles of mineralized organic material, comprising crystals of an anion and a cation, which crystals cover the organic material. Such a bone deficiency is, e.g., loss of bone matter, naturally or artificial, e.g. by surgery. The inventive particle compositions can be used as a bone void filler in any procedures where such a material is useful. However, the materials can also be used in soft tissue regeneration in selected indications where the material properties are useful.

In another aspect the present invention relates to a method of treating a bone deficiency comprising applying to the site of deficiency particles according to the present invention or a pharmaceutical composition according to the present invention.
In another aspect the present invention relates to a method for treating bleeding of a bone lesion comprising applying to the site to be treated particles according to the present invention or a pharmaceutical composition according to the present invention, and a coagulation factor, e.g. thrombin.

The present invention is further exemplified by the following examples without being limited thereto.

The following abbreviations are used:
H₂O water
IR infrared
min minutes
RT room temperature

### SEM Scanning Electron Microscope

### EXAMPLES

### Example 1: Mineralized Gelatin Preparation under Various Conditions

A first solution containing 0.125 M of calcium acetate monohydrate and 0.04 M Tris(hydroxymethyl aminomethane hydrochlorid) (TRIS-HCl) as buffer in double-distilled water (ddH₂O)is prepared. As antibacterial agent sodium azide is used at a concentration of 0.02 wt %. The pH of the solution is adjusted to 7.3 using 1N solution of NaOH. A second solution containing 0.1 M of disodium hydrogen phosphate dihydrate, 0.04M TRIS and 0.02 wt % of sodium azide is prepared in ddH₂O. A final pH of the solution is adjusted to 7.3 using 1 N solution of HCl. Both solutions are used in a mineralization process without filtration.

In a test tube (15 ml) 0.6 g of dried cross-linked gelatin are suspended in 12 ml of calcium solution. Particles are treated for 30 min under constant agitation at RT. Gelatin particles are collected by centrifugation and washed once with ddH₂O. H₂O is removed by centrifugation and wet gelatin particles are treated in the phosphate solution. Particles are kept in solution for 30 min under constant agitation at RT. Particles are separated from solution by centrifugation and washed once with ddH₂O. The procedure described above (treatment with calcium solution, washing with H₂O, treatment in phosphate solution, washing with H₂O) is considered as one mineralization cycle.

After a specific number of mineralization cycles the mineralized gelatin is dried in a vacuum oven at RT until no further mass loss is observed.

Repeating the mineralization process many times increases the amount of mineral deposit in and on the gelatin. This is confirmed by thermogravimetry measurements. The amount of inorganic component after 4, 8, 12, 16, 20 and 24 cycles is 22, 38, 44, 50, 56 and 67 wt %, respectively.

SEM reveals deposit of needle-like tiny crystals on the surface of the gelatin particles. This inorganic component growing on/in the gelatin particles is identified by IR spectroscopy in DRIFT mode as calcium phosphate salt with adsorption peaks at 560, 601, 962 cm⁻¹ characteristic for hydroxyapatite.

### Example 2: Effect of Temperature on Mineralization of Gelatin

The mineralization process of gelatin is carried out as described in Example 1 with the exception that the process is carried out at the following temperatures: 4° C., RT (22-25° C.)) and 50° C. The composition of calcium and phosphate solutions and the type and concentration of salts are the same as in example 1.

Incubation of gelatin particles in specific solution is carried out at 4° C., RT or 50° C. In all experiments washing is done at RT.

Samples characterization indicates that higher temperature enhances the mineralization process. After 16 mineralization cycles there are 43, 50, 59 wt % of inorganic material when gelatin is mineralized at 4° C., RT, and 50° C., respectively. SEM micrographs show that when gelatin is mineralized at a higher temperature (50° C.), the crystals tend to aggregate and form more compact structures of the inorganic material. In this case it is difficult to recognize the needle-like morphology of crystals that are characteristic for inorganic material grown at RT. IR spectroscopy reveals that the major component of inorganic phase grown at 4°, RT, and 50° C. is hydroxyapatite.

### Example 3: Strontium Mineralization

A first solution containing 0.125 M of strontium nitrate and 0.6 mM Tris(hydroxymethyl aminomethane hydrochlorid) (TRIS-HCl) as buffer in double-distilled water (ddH₂O) is prepared. The pH of the solution is adjusted to 7.4 using 0.1N solution of NaOH. A second solution containing 0.06 M of disodium hydrogen phosphate dihydrate and 0.015 M of sodium dihydrogenphosphate hydrate is prepared in ddH₂O. A final pH of the solution is adjusted to 7.4. As antibacterial agent silver acetate is used at a concentration of 1 ppm (ion concentration) but only in phosphate solution. Both solutions are used in the mineralization process without filtration.

In a test tube (50 ml) 2 g of dried cross-linked gelatin are suspended in 40 ml of calcium solution. Particles are treated in this solution for 30 min under constant agitation at RT. After this time the gelatin particles are collected by centrifugation and washed twice with ddH₂O. H₂O is removed by centrifugation and wet gelatin particles are treated in the phosphate solution. Particles are kept in this solution for 30 min under constant agitation at RT. After this time particles are separated from the solution by centrifugation, washed twice with ddH₂O. The procedure described above (treatment with calcium solution, washing in water, treatment in phosphate solution, washing with H₂O) is considered as one mineralization cycle. All procedures are carried out at RT.

After a specific number of mineralization cycles the mineralized gelatin is dried in a vacuum oven at RT until no further mass loss is observed.

Repeating the mineralization process many times increases the amount of mineral deposit in and on the gelatin. This is confirmed by thermogravimetry measurements. The amount of inorganic component after 8, 12, 16, and 20 cycles is 29, 32, 38, 41 wt %, respectively.

SEM reveals deposit of needle-like tiny crystals on surface of the gelatin particles. This inorganic component growing on/in the gelatin particles is identified by IR spectroscopy in MIRacle ATR mode as strontium phosphate salt with main adsorption peaks at 537, 594, 882, 926, 1003, 1055, 1133 cm⁻¹.

1. A process for mineralizing a particulate organic material comprising
a) providing particles of a non-porous, swellable organic material,
b) providing a solution of containing at least one cation,
c) providing a solution of containing at least one anion,
d) in any subsequent order:
contacting said particles with said solution of the cation for a time period allowing adsorption of said cation on said organic material,
contacting said particles with said solution of the anion for a time period allowing adsorption of said anion on said organic material,
thereby obtaining said mineralized particulate organic material.

2. The process according to claim 1, wherein the particles are contacted with the solution of the cation before being contacted with the solution of the anion.

3. The process of claim 1, wherein any one or both of the contacting steps of step d) are repeated at least 10 times.

4. The process according to claim 1 further comprising a washing step, wherein unbound anion is removed from the particles after contacting with said solution of the anion.

5. The process according to claim 1 further comprising a washing step, wherein unbound cation is removed from the particles after contacting with said solution of the cation.

6. The process according to claim 1, wherein the cation is at least divalent.

7. The process according to claim 1, wherein the cation is selected from calcium, magnesium, barium, strontium, zinc, nickel, manganese, copper, selenium, iron, silicon, vanadium, silver, gadolinium, bismuth, or combinations thereof.

8. The process according to claim 1, wherein the anion is selected from phosphate, carbonate, sulfate, oxalate, silicate, fluoride, citrate, or combinations thereof.

9. The process according to claim 1, wherein the organic material comprises collagen, gelatin, fibrin, dextrane, agarose, hyaluronic acid, cellulose, fibronectin, a synthetic polymer or a derivative thereof.

10. The process according to claim 1, wherein the particles have an average diameter of between 100 nm and 2000 μm.

11. The process according to claim 1 further comprising dispersing the particles in a fluid.

12. Particles of mineralized organic material obtainable by a method of claim 1.

13. Pharmaceutical composition comprising the particles according to claim 12 and at least one additive selected from the group consisting of collagen, gelatin,. fibrin, dextrane, agarose, hyaluronic acid, cellulose, fibronectin, a synthetic polymer or a derivative thereof.

14. A method of treating a bone deficiency comprising applying to the site of deficiency particles according to claim 12.

15. A method for treating bleeding of a bone lesion comprising applying to the site to be treated particles according to claim 12 and a coagulation factor.
